(19) United States
(12) Patent Application Publication
(10) Pub. No.: US 2011/0206693 A1
(43) Pub. Date: Aug. 25, 2011

(54) LYSINE RIBOSWITCH AND COMPOSITIONS AND USES THEREOF

(76) Inventors: Robert Batey, Boulder, CO (US); Andrew Garst, Boulder, CO (US)

(21) Appl. No.: 12/937,771

(22) PCT Filed: Apr. 14, 2009
(86) PCT No.: PCT/US09/02387

§ 371 Date: Apr. 28, 2011

Related U.S. Application Data
(60) Provisional application No. 61/044,810, filed on Apr. 14, 2008.

(51) Int. Cl. A61K 39/395; C12N 1/20; C07H 21/02; C07K 16/12; C12Q 1/68; G06G 7/60

(52) U.S. Cl. 424/164.1; 435/252.1; 536/23.1; 536/24.5; 530/389.5; 435/6.1; 703/11

(57) ABSTRACT

Embodiments herein provide for lysine riboswitches and analogs thereof, and methods for using the same. In certain embodiments, test compounds are identified that associate with lysine riboswitches. In other embodiments, test compounds found to associate with lysine can be used to increase or decrease gene expression of Gram-negative bacterial organisms.

This application claims the benefit of U.S. Provisional Application No. 61/044,810, filed Apr. 14, 2008, the contents of which application are incorporated herein by reference.

The U.S. Government has a paid-up license in this invention and the right in limited circumstances to require the patent owner to license others on reasonable terms as provided for by the terms of Grant No. R-01 GM073850-01 awarded by the National Institutes of Health.

### FIELD

The present invention relates to compositions and methods of use thereof related to lysine riboswitch.

### BACKGROUND

Riboswitches are regulatory elements found within the 5′-untranslated regions (5′-UTRs) of many bacterial mRNAs. Riboswitches control gene expression in a cis-fashion through their ability to directly bind a specific small molecule metabolite. Ligand recognition is effected by the first domain of the riboswitch, termed the aptamer domain, while the second, the expression platform, transduces the binding event into a regulatory switch. The switch includes an RNA element that can adapt to one of two mutually exclusive secondary structures. One of these structures is a signal for gene expression to be “on” and the other conformation turns the gene “off” In Bacillus subtilis and other gram positive bacteria, it is believed riboswitches control greater than 4% of all genes, many of which are important for key pathways controlling amino acid, nucleotide and cofactor metabolism.

Currently, there at least 20 distinct families of riboswitches that have been identified that recognize a diverse set of metabolites including nucleobases, sugars, vitamin cofactors, amino acids and metal ions. The lysine binding riboswitch is of particular importance for several reasons. While in vitro selection methods are capable of raising artificial aptamers to equally diverse set of compounds, one of the few compounds that has failed to yield a corresponding aptamer is lysine. Thus, how a natural RNA has managed to achieve specific recognition of a compound that bears no chemical similarity will provide new insights into the range of ligand binding by aptamers. Second, the lysine riboswitch has been the focus of studies involving the potential of riboswitches as targets of antimicrobial agents.

Riboswitch aptamer domains are controlled by a diverse set of metabolites. In one example amino acid metabolism in various Bacillus species is controlled by three known riboswitches: glycine, lysine and S-adenosylmethionine (SAM). Each has a distinct aptamer domain, but lysine is one of the few molecules for which an aptamer has failed to be raised. In order to further identify bacterial regulation of the lysine riboswitch, a need exists for crystallizing the structure of this riboswitch and identifying interactions of these riboswitches with its ligand.

A need exist to better control bacterial growth, such as Gram negative bacterial growth, and generate effective treatments against bacterial infections. Embodiments herein fulfill this need.

### SUMMARY

Embodiments herein provide for methods of identifying a compound that associates with a lysine riboswitch including modeling at least a portion of the atomic structure depicted in FIGS. 7A and 7B with a test compound; and determining the interaction between the test compound and the lysine riboswitch structure. Embodiments herein concern compositions and methods for controlling bacteria growth through a common regulatory element. Certain embodiments herein, identify nucleotides that play a role in lysine binding to lysine riboswitches throughout bacteria. Other embodiments concern developing novel antimicrobial compounds that bind the RNA to reduce or inhibit lysine metabolism in bacteria. It is contemplated herein that antimicrobial compounds may be used to reduce, ameliorate, prevent or treat a subject having or suspected of developing a bacteria-caused disorder.

Certain embodiments herein concern crystalline atomic structures of lysine riboswitches. In accordance with the methods, the structures may also be used for modeling and assessing the interaction of a riboswitch with a binding ligand.

In other embodiments herein, a compound may be identified that associates with the lysine riboswitch and reduces bacterial gene expression or associates with the lysine riboswitch and induces bacterial gene expression. In a more particular embodiment, a bacteria can be a Gram negative bacteria. In accordance with these embodiments, atomic coordinates of the atomic structure can include at least a portion of the atomic coordinates listed in Table 1 for atoms depicted in FIGS. 7A and 7B wherein said association determination step can include determining a minimum interaction energy, a binding constant, a dissociation constant, or a combination thereof, for the test compound in the model of the lysine riboswitch. In some particular embodiments, an association determination step can include determining the interaction of the test compound with a nucleotide of lysine riboswitch including G9, C76, G77, G111, U137 or combinations thereof. In other embodiments, an association determination step can include determining the interaction of the test compound with a lysine moiety including a carboxylate group and two amino groups and combinations thereof. Alternatively, in a more particular embodiment, the association determination step can include determining the interaction of the test compound with a nucleotide of lysine riboswitch depicted in FIGS. 7A and 7B including G9, C76, G77, G111, U137 or a combination thereof, for example by determining the interaction of nucleotides around the binding pocket, e.g. G8, C76, G77, A78, G111, U137, G138, A151, G152. Other embodiments contemplated herein include an association determination step of identifying the interaction of the test compound with a P1 helix region or 5-way junction (identified herein) of the lysine riboswitch. Yet other embodiments contemplated herein can include an association determination step including determining the interaction of the test compound within the 5-way junction of the lysine riboswitch. Further embodiments concern an association determination step including determining the interaction of the test compound with the P1 helix and/or J2/3 of the lysine riboswitch. In accordance with these embodiments, further interaction of a test compound may be analyzed in the flanking first base pairs of the P2 and P4 helices.

Bacterial cells contemplated of use in the methods and compositions herein include, but are not limited to, Gram negative species, for example, proteobacteria including Escherichia coli, Salmonella, and other Enterobacteriaceae, Pseudomonas, Moraxella, Helicobacter, Stenotrophomonas, Bdellovibrio, acetic acid bacteria, Legionella and many others. Other groups of Gram-negative bacteria include the cyanobacteria, spirochaetes, green sulfur and green non-sulfur bacteria. Medically relevant Gram-negative cocci include organisms, that cause staph infections (Staphylococcus aureus), Medically relevant Gram-negative bacilli include, but are not limited to those that primarily cause respiratory problems (Hemophilus influenzae, Klebsiella pneumoniae, Legionella pneumophila, Pseudomonas aeruginosa), cholera (Vibrio cholerae), principally urinary problems (Escherichia coli, Proteus mirabilis, Enterobacter cloacae, Serratia marcescens), tetanus (Clostridium tetani), and usually gastrointestinal problems (Helicobacter pylori, Salmonella enteritidis, Salmonella typhi, Shigella flexneri). Nosocomial gram negative bacteria can include Acinetobacter baumanii, which cause bacteremia, secondary meningitis, and ventilator-associated pneumonia. Medically relevant coccoid bacteria known to contain the lysine riboswitch include, but are not limited to, Bortedella pertusis and Bortedella bronchiseptica that causes whopping cough. One Gram-positive bacillus of medical relevance that contains known lysine riboswitches is Bacillus anthracis, the cause of anthrax, a known bioterror weapon.

In certain embodiments, a lysine riboswitch disclosed herein can include one or more of the nucleotides listed herein where the nucleotide can be modified. In certain embodiments, the one or more modified nucleotides are selected from the group consisting of G9, C76, G77, G111, U137 or combinations thereof, or from the group consisting of nucleotides around the binding pocket, e.g. G8, C76, G77, A78, G111, U137, G138, A151, G152. In particular embodiments, the modified nucleotide of the lysine riboswitch can increase gene expression in a bacterial cell. For example, a test compound that contains a modified nucleotide may induce expression of a gene that is deleterious to a bacterial cell. In other embodiments, the modified nucleotide can decrease gene expression in a cell. For example, a test compound that contains a modified nucleotide may reduce expression of a gene that is necessary for survival of a bacterial cell. In certain particular embodiments, the modified nucleotide decreases sulfur production in a cell.

Embodiments of the present invention concern a test compound that associates with at least a portion of the lysine riboswitch atomic structure depicted in at least one of FIGS. 7A and/or 7B. In accordance with these embodiments, the association can include association with at least one of nucleotides G9, C76, G77, G111, U137 or combinations thereof, or with nucleotides around the binding pocket, e.g. one or more of G8, C76, G77, A78, G111, U137, G138, A151, G152, wherein the composition is capable of modifying the lysine riboswitch activity of a bacterial organism by either inducing or reducing gene expression.

Certain embodiments concern compositions including, all of the 80 percent or more conserved nucleotides of the lysine riboswitch depicted in FIG. 5A and 80% or greater, or 90% or greater or 95% or greater of the nucleotides depicted outside of the conserved region. One particular embodiment includes a composition of all 80 percent or more conserved nucleotides of the lysine riboswitch depicted in FIG. 5A and all of the nucleotides depicted outside of the conserved region.

In one embodiment, the atomic coordinates of the atomic structure comprise the atomic coordinates listed in Table 1 for atoms depicted in FIGS. 1C, 3D and 7A.

Yet in another embodiment, the interaction determination step can include determining a minimum interaction energy, a binding constant, a dissociation constant, or a combination thereof, for the test compound in the model of the lysine riboswitch.

Still in other embodiments, the interaction determination step and test compound identification can include determining the interaction of the test compound with a nucleotide of lysine riboswitch comprising G9, C76, G77, G111, U137 or combinations thereof, or e.g. comprising nucleotides around the binding pocket, e.g. one or more of G8, C76, G77, A78, G111, U137, G138, A 151. Within this embodiment, the interaction determination step can include determining the interaction of the test compound with a nucleotide of lysine riboswitch comprising G9, C76, G77, G111, U137 or combinations thereof, or e.g. comprising nucleotides around the binding pocket, e.g. one or more of G8, C76, G77, A78, G111, U137, G138, A151. In addition, the test compound that effectively interacts with one or more of the above mentioned nucleotides can be identified and expanded for use in targeting bacterial organisms disclosed herein.

Another aspect of the present invention provides, a method of regulating a gene in a cell by modulating an mRNA, said method comprising administering a lysine riboswitch modulating compound to the cell to modulate the lysine riboswitch activity of the mRNA. In certain embodiments, the gene expression is stimulated, while in other embodiments the gene expression is inhibited. Within certain embodiments where the gene expression is inhibited, the lysine riboswitch modulating compound forms a complex with the lysine riboswitch, thereby preventing the mRNA from forming an antiterminator element.

Certain embodiments include a compound that associates with one or more of the contact nucleotides and modulates the activity of the lysine riboswitch. In one particular embodiment, a compound capable of associating with one or more of the contact nucleotides may be capable of reducing sulfur metabolism in an organism having a lysine or lysine like riboswitch. In accordance with these embodiments, compounds of the present invention may be used to reduce infection caused by, or as a treatment for infection caused by an organism contemplated herein. In certain embodiments target organisms include bacteria. Bacteria contemplated herein include, but are not limited to Gram-negative bacterial organisms.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIGS. 1A-1C represent exemplary structures of a lysine riboswitch.

FIGS. 2A-2D represent exemplary tertiary structural elements in a lysine riboswitch.

FIGS. 3A-3D represent exemplary lysine recognition by the five-way junction.

FIG. 4 represents an exemplary schematic of an experimental density map of lysine riboswitch.

FIGS. 5A-5B represent an exemplary schematic of a ligand binding pocket of lysine riboswitch: (A) Final 2Fo-Fc map contoured at 1.0σ around the nucleotide residues that define the binding pocket and lysine, and (B) Simulated annealing omit map in which residues 76, 77, 111 were omitted along with lysine.

FIG. 6 represents an exemplary schematic of a mobility shift assay of riboswitches with protein L7Ae.

FIGS. 7A and 7B represent schematics of exemplary superposition of free and bound lysine riboswitch. (A) superpositioning of the free and bound structures of the lysine riboswitch using the Theseus alignment program (D. L. Theobald, D. S. Wuttke, Bioinformatics 22, 2171 (Sep. 1, 2006), incorporated herein by reference in its entirety). (B) An exemplary map of the estimated variance between the two structures in atomic coordinates between the two structures.

FIG. 8 represents a schematic of some details of superposition of the binding pocket of lysine riboswitch.

### DEFINITIONS

As used herein, “a” or “an” may mean one or more than one of an item.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that molecules, test compounds, samples, concentrations, times and other specific details may be modified through routine experimentation. In some cases, well known methods or components have not been included in the description.

Embodiments herein provide for compositions and methods concerning lysine riboswitch and lysine riboswitch-like molecules.

Riboswitch aptamer domains are controlled by a diverse set of metabolites. Amino acid metabolism in various Bacillus species is controlled by three known riboswitches: glycine, lysine and S-adenosylmethionine (SAM). Each has a distinct aptamer domain that has evolved to specifically recognize a specific ligand. Currently, there at least 15-20 distinct families of riboswitches that have been identified that recognize a diverse set of metabolites including nucleobases, sugars, vitamin cofactors, amino acids and metal ions. The lysine binding riboswitch is of particular importance for several reasons. While in vitro selection methods are capable of raising artificial aptamers to equally diverse set of compounds, one of the few compounds that has failed to yield a corresponding aptamer is lysine. Thus, how a natural RNA has managed to achieve specific recognition of a compound that bears no chemical similarity will provide new insights into the range of ligand binding by aptamers. Second, the lysine riboswitch has been the focus of studies involving the potential of riboswitches as targets of antimicrobial agents. The combination of the ability of these RNAs to already bind small molecules coupled with the fact that RNA is already a well-validated target of antibiotics makes riboswitches a significant new avenue for the development of new therapeutics.

Non-coding small RNAs and mRNA sequences play a central role in genetic regulation and are involved in virtually every aspect of the maintenance and transmission of genetic information. One common form of riboregulation is the riboswitch, a noncoding element that exerts genetic control in a cis-fashion via its ability to specifically bind a cellular metabolite that in turn directs formation of one of two mutually exclusive mRNA secondary structures. Depending upon placement within the mRNA, they control transcription or translation in bacteria, and alternative splicing or mRNA stability in eukarya. Thus, these sequences are extraordinarily versatile regulatory elements.

Certain embodiments herein concern compositions and methods for selecting and identifying compounds that can activate, deactivate or block lysine riboswitch. Activation or deactivation of a lysine riboswitch refers to the change in state of the riboswitch upon binding of the compound of interest, a test compound. The term trigger molecule is used herein to refer to molecules and compounds that can activate the lysine riboswitch.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a ribo switch refers to a condition or state of the riboswitch where the presence of the trigger molecule does not activate the riboswitch.

In certain particular embodiments, methods of identifying a compound that interact with a lysine riboswitch include modeling the atomic structure of the lysine riboswitch with a test compound and determining if the test compound interacts with the lysine riboswitch. In accordance with these embodiments, the atomic contacts of the lysine riboswitch and test compound can be determined by means known in the art. Further, analogs of a compound known to interact with a lysine riboswitch can be generated by analyzing the atomic contacts, for example the contacts that interact with ligand binding, then optimizing the atomic structure of the analog to maximize interaction. In certain embodiments, these methods can be used in a high throughput screen.

Other embodiments concern methods for identifying compounds that block a riboswitch. For example, an assay can be performed for assessing the induction or inhibition of lysine riboswitch in the presence of a test compound.

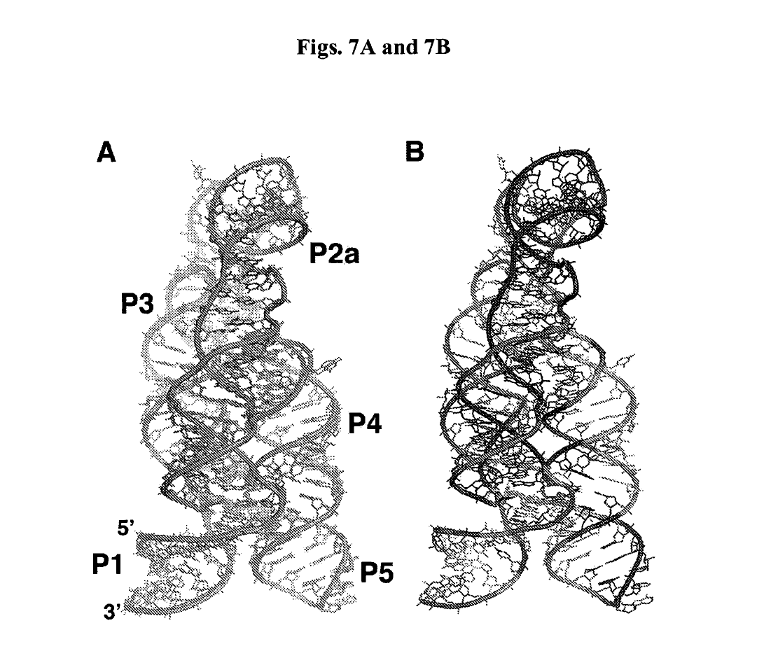

Some embodiments herein concern compositions and methods for identifying a test compound for significantly reducing the activity or inactivating a lysine riboswitch by binding the test compound to at least a portion of the atomic structure represented in FIGS. 7A and 7B. In accordance with these embodiments, activity of the lysine riboswitch can be measured by any methods known in the art. For example, the activity of the riboswitch can be measured in the presence or absence of a test compound in order to identify the efficiency of the test compound to reduce the activity of or inactivate the lysine riboswitch. Inactivation of a riboswitch in this manner can result from, for example, an alteration that prevents lysine molecule from binding; that prevents the change in state of the lysine riboswitch upon binding of lysine; or the binding of the test compound interferes with ligand interaction or prevents the change in state of the lysine riboswitch.

In other embodiments, a test compound that activates a lysine riboswitch can be identified. For example, test compounds that activate a riboswitch can be identified by bringing into contact a test compound and a lysine riboswitch including at least a portion of the lysine riboswitch of FIGS. 7A and 7B and assessing activation of the riboswitch. Activation of a lysine riboswitch can be assessed in any suitable manner. For example, activation of the lysine riboswitch can be measured by expression level of or modification of the expression level of a reporter gene in the presence or absence of the test compound. Examples of a reporter gene include, but are not limited to, beta-galactosidase, luciferase or green-fluorescence protein.

The lysine riboswitch is known to regulate multiple operons in a number of bacteria. Bacterial cells contemplated herein include, but are not limited to, Gram negative species, for example, proteobacteria including Escherichia coli, Salmonella, and other Enterobacteriaceae, Pseudomonas, Moraxella, Helicobacter, Stenotrophomonas, Bdellovibrio, acetic acid bacteria, Legionella and many others. Other groups of Gram-negative bacteria include the cyanobacteria, spirochaetes, green sulfur and green non-sulfur bacteria. Medically relevant Gram-negative cocci include organisms, that cause staph infections (Staphylococcus aureus), Medically relevant Gram-negative bacilli include, but are not limited to those that primarily cause respiratory problems (Hemophilus influenzae, Klebsiella pneumoniae, Legionella pneumophila, Pseudomonas aeruginosa), cholera (Vibrio cholerae), principally urinary problems (Escherichia coli, Proteus mirabilis, Enterobacter cloacae, Serratia marcescens), tetanus (Clostridium tetani), and usually gastrointestinal problems (Helicobacter pylori, Salmonella enteritidis, Salmonella typhi, Shigella flexneri). Nosocomial gram negative bacteria can include Acinetobacter baumanii, which cause bacteremia, secondary meningitis, and ventilator-associated pneumonia. One Gram-positive bacillus of medical relevance that contains known lysine riboswitches is Bacillus anthracis, the cause of anthrax, a known bioterror weapon.

### Organization of Riboswitch RNAs

Structural probing studies demonstrate that bacterial riboswitch elements are composed of two domains: a natural aptamer that serves as the ligand-binding domain, and an ‘expression platform’ that interfaces with RNA elements that are involved in gene expression. Structural probing investigations suggest that the aptamer domain of most riboswitches adopts a particular secondary- and tertiary-structure fold when examined independently, that is essentially identical to the aptamer structure when examined in the context of the entire 5′ leader RNA. This implies that, in many cases, the aptamer domain is a modular unit that folds independently of the expression platform.

The ligand-bound or unbound status of the aptamer domain is interpreted through the expression platform, which is responsible for exerting an influence upon gene expression. The aptamer domains are highly conserved amongst various organisms, whereas the expression platform varies in sequence, structure, and in the mechanism by which expression of the appended open reading frame is controlled.

Aptamer domains for riboswitch RNAs typically range from ˜70 to 170 nucleotides in length. Some aptamer domains, when isolated from the appended expression platform, exhibit improved affinity for the target ligand over that of the intact riboswitch (˜10 to 100-fold). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### Riboswitch Regulation

Bacteria primarily use two methods for termination of transcription. Certain genes incorporate a termination signal that is dependent upon the Rho protein, while others make use of Rho-independent terminators (intrinsic terminators) to destabilize the transcription elongation complex. The latter RNA elements are composed of a GC-rich stem-loop followed by a stretch of 6-9 uridyl residues. Intrinsic terminators are widespread throughout bacterial genomes, and are typically located at the 3′-termini of genes or operons. Interestingly, an increasing number of examples are being observed for intrinsic terminators located within 5′-UTRs.

In certain examples, RNA polymerase responds to a termination signal within the 5′-UTR in a regulated fashion. Under certain conditions, the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances an RNA is generally required for receiving a particular intracellular signal and subsequently stabilizing one of the RNA conformations. Riboswitches offer a direct link between RNA structure modulation and the metabolite signals that are interpreted by the genetic control machinery.

Certain mRNAs involved in thiamine biosynthesis bind to thiamine (vitamin B₁) or its bioactive pyrophosphate derivative (TPP) without the participation of protein factors. The mRNA-effector complex adopts a distinct structure that sequesters the ribosome-binding site and leads to a reduction in gene expression. This metabolite-sensing mRNA system provides an example of a genetic “riboswitch” (referred to herein as a riboswitch) whose origin might predate the evolutionary emergence of proteins. It has been discovered that the mRNA leader sequence of the btuB gene of Escherichia coli can bind coenzyme B₁₂ selectively, and that this binding event brings about a structural change in the RNA that is important for genetic control. It was also discovered that mRNAs that encode thiamine biosynthetic proteins also employ a riboswitch mechanism.

Although certain specific natural riboswitches such as lysine riboswitch was one of the first examples of mRNA elements that control genetic expression by metabolite binding, it is suspected that this genetic control strategy may be widespread in biology. If these metabolites were being biosynthesized and used before the advent of proteins, then certain riboswitches might be modern examples of the most ancient form of genetic control. A search of genomic sequence databases has revealed that sequences corresponding to the TPP aptamer exist in organisms from bacteria, archaea and eukarya—largely without major alteration. Although new metabolite-binding mRNAs are likely to emerge as evolution progresses, it is possible that the known riboswitches are molecular fossils from the RNA world.

In certain embodiments, it is contemplated that a Lysine Reporter system can be used to assess whether a test compound activates or inactivates the lysine riboswitch. In some embodiments, an in vitro selection protocol can be designed for example to assess whether a test compound activates or deactivates the lysine riboswitch. Some embodiments herein concern binding of the ligand can be monitored by a mobility-shift assay, known in the art, to discern free and bound RNA, providing a basis for selection of binding-competent RNAs. Ligand binding to the RNA can cause a conformational and/or secondary structural change in the RNA that can result in a change in its migration in a native polyacrylamide gel.

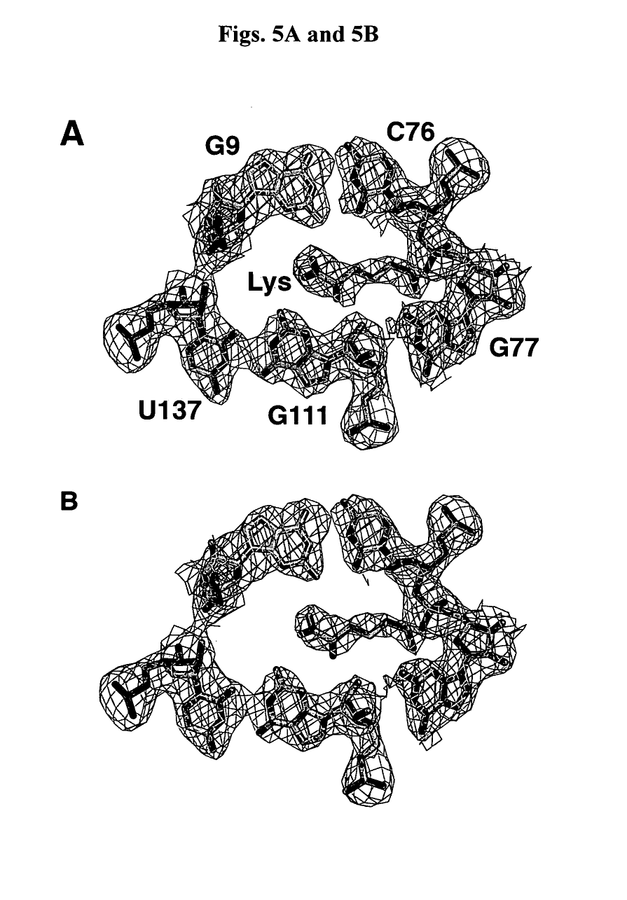

In certain embodiments, a detectible tag can be incorporated into the lysine riboswitch. In accordance with these embodiments, a test compound can be placed in contact with the lysine riboswitch and the interaction of the test compound and the lysine riboswitch assessed by measuring the presence or absence of a detectible tag. In certain particular examples, a detectible tag may be undetectable in the presence of a test compound thereby quenching the signal. This mechanism can be adapted to existing lysine riboswitches, as this method can take advantage of assessing a ligand-mediated interaction of the lysine riboswitch. In some embodiments, a detectible tag can be placed within the ligand interaction region. In other embodiments, a detectible tag can be placed on any one of ligand binding nucleic acids, including but not limited to, G9, C76, G77, G111, U137 or combinations thereof, or e.g. comprising nucleotides around the binding pocket, e.g. one or more of G8, C76, G77, A78, G111, U137, G138, A151, of FIG. 7A or FIG. 7B or FIG. 5A of the lysine riboswitch. In these examples, a test compound can be combined with a lysine riboswitch depicted FIG. 7A or FIG. 7B and a detectible signal on the lysine riboswitch quenched when the test compound binds to at least one of the ligand-binding nucleic acids indicated above. In one example, a florescent tag molecule can be positioned in RNA adjacent to the binding site of lysine and binding can be monitored via a change in fluorescence of a reporter gene.

In other embodiments, control compounds can be used to assess interaction of the test compound compared to a known compound that interacts with a lysine riboswitch. To use riboswitches to report ligand binding by analyzing for a detectible tag, the appropriate construct can be determined empirically. The optimum length and composition of a test compound and its binding site on the riboswitch can be assessed systematically to result in the highest ligand binding region interaction possible. The validity of the assay can be determined by comparing apparent relative binding affinities of different lysine analogs, lysine antibodies or other lysine binding agents to a particular test compound (determined by the presence or level of detectible signal generation of the tag) to the binding constants determined by standard in-line probing.

In other embodiments, interaction of a test compound with at least a portion of the atomic structures depicted in FIG. 7A or FIG. 7B may be assessed by measuring uptake and/or synthesis of lysine in a bacterial test cell system (e.g., cultures of B. subtilus). In accordance with these embodiments, a test compound confirmed to interact with at least a portion of the atomic structures depicted in FIG. 7A or FIG. 7B can be synthesized and/or purified for future use. In one example use, the test compound may be placed in contact with lysine riboswitch and the uptake and/or metabolism of lysine can be measured. If a test compound is found to effectively block these functions, the test compound may be a candidate for use in inhibiting bacterial expansion or eliminating bacteria within a subject or a system.

It is contemplated herein that test compounds capable of associating with the atomic structures depicted in FIG. 7A or FIG. 7B or FIG. 5A may be a nucleic acid molecule, a small molecule, an antibody, a pharmaceutical agent, small peptide, peptide mimetic, nucleic acid mimetic, modified saccharide or aminoglycoside. Preferred test compound compositions would be small molecule mimetics of lysine or nucleic acid mimetics that build off of the adenosine moiety of lysine.

### Kits

In still further embodiments, kits for methods and compositions described herein are contemplated. In one embodiment, the kits have a point-of care application, for example, the kits may have portability for use at a site of suspected bacterial outbreak. In another embodiment, a kit for treatment of a subject having a bacterial-induced infection is contemplated. In accordance with this embodiment, the kit may be used to reduce the bacterial infection of a subject.

The kits may include a container means. Any of the kits will generally include at least one vial, test tube, flask, bottle, syringe or other container means, into which the testing agent, may be preferably and/or suitably aliquoted. Kits herein may also include a means for comparing the results such as a suitable control sample such as a positive and/or negative control.

### Nucleic Acids

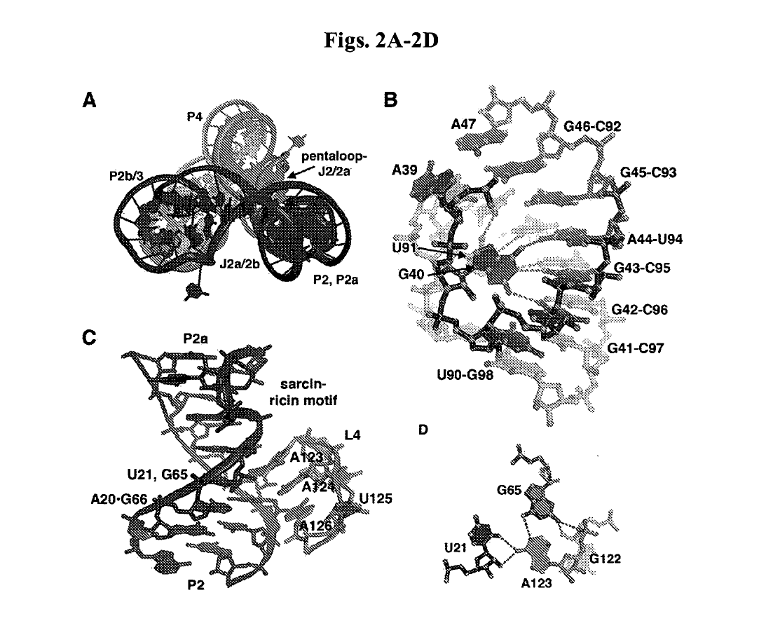

In various embodiments, isolated nucleic acids may be used as test compounds for binding the atomic structure depicted in FIG. 2 or 3 or 5. The isolated nucleic acid may be derived from genomic RNA or complementary DNA (cDNA). In other embodiments, isolated nucleic acids, such as chemically or enzymatically synthesized DNA, may be of use for capture probes, primers and/or labeled detection oligonucleotides.

A “nucleic acid” includes single-stranded and double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

### Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a portion of a lysine riboswitch. For example, a multi-cloning site comprising one or more endonuclease restriction sites may be added. A nucleic acid may be attached to a vector, adapter, or linker for cloning of a nucleic acid. Additional sequences may be added to such cloning and sequences to optimize their function, to aid in isolation of the nucleic acid, or to improve the introduction of the nucleic acid into a cell. Use of cloning vectors, expression vectors, adapters, and linkers is well known in the art.

### Recombinant Methods for Constructing Nucleic Acids

Isolated nucleic acids may be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods may be used.

### Nucleic Acid Screening and Isolation

Bacterial RNA or cDNA may be screened for the presence of an identified genetic element of interest using a probe based upon one or more sequences. Various degrees of stringency of hybridization may be employed in the assay. As the conditions for hybridization become more stringent, there must be a greater degree of complementarity between the probe and the target for duplex formation to occur. The degree of stringency may be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, the degree of complementarity can optimally be about 100 percent; but in other embodiments, sequence variations in the RNA may result in <100% complementarity, <90% complimentarity probes, <80% complimentarity probes, <70% complimentarity probes or lower depending upon the conditions. In certain examples, primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids may be completely complementary to a target sequence or may exhibit one or more mismatches.

### Nucleic Acid Amplification

Nucleic acids of interest may also be amplified using a variety of known amplification techniques. For instance, polymerase chain reaction (PCR) technology may be used to amplify target sequences directly from bacterial RNA or cDNA. PCR and other in vitro amplification methods may also be useful, for example, to clone nucleic acid sequences, to make nucleic acids to use as probes for detecting the presence of a target nucleic acid in samples, for nucleic acid sequencing, or for other purposes.

### Synthetic Methods for Constructing Nucleic Acids

Isolated nucleic acids may be prepared by direct chemical synthesis by methods such as the phosphotriester method, or using an automated synthesizer. Chemical synthesis generally produces a single stranded oligonucleotide. This may be converted into double stranded DNA by hybridization with a complementary sequence or by polymerization with a DNA polymerase using the single strand as a template. While chemical synthesis of DNA is best employed for sequences of about 100 bases or less, longer sequences may be obtained by the ligation of shorter sequences.

### Covalent Modification of Nucleic Acids

A variety of cross-linking agents, alkylating agents and radical generating species may be used to bind, label, detect, and/or cleave nucleic acids. In addition, covalent crosslinking to a target nucleotide using an alkylating agent complementary to the single-stranded target nucleotide sequence can be used. A photoactivated crosslinking to single-stranded oligonucleotides mediated by psoralen can be used. Use of N4, N4-ethanocytosine as an alkylating agent to crosslink to single-stranded oligonucleotides has also been disclosed. Various compounds to bind, detect, label, and/or cleave nucleic acids are known in the art.

### Nucleic Acid Labeling

In various embodiments, tag nucleic acids may be labeled with one or more detectable labels to facilitate identification of a target nucleic acid sequence bound to a capture probe on the surface of a microchip. A number of different labels may be used, such as fluorophores, chromophores, radio-isotopes, enzymatic tags, antibodies, chemiluminescent, electroluminescent, affinity labels, etc. One of skill in the art will recognize that these and other label moieties not mentioned herein can be used. Examples of enzymatic tags include urease, alkaline phosphatase or peroxidase. Colorimetric indicator substrates can be employed with such enzymes to provide a detection means visible to the human eye or spectrophotometrically. A well-known example of a chemiluminescent label is the luciferin/luciferase combination.

In preferred embodiments, the label may be a fluorescent, phosphorescent or chemiluminescent label. Exemplary photodetectable labels may be selected from the group consisting of Alexa 350, Alexa 430, AMCA, aminoacridine, BODIPY 630/650, BODIPY 650/665, BODIPY-FL, BODIPY-R6G, BODIPY-TMR, BODIPY-TRX, 5-carboxy-4′,5′-dichloro-2′,7′-dimethoxy fluorescein, 5-carboxy-2′,4′,5′,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FAM, dansyl chloride, Fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin C, phycocyanin R, thiamine, phycoerythrocyanin, phycoerythrin R, REG, Rhodamine Green, rhodamine isothiocyanate, Rhodamine Red, ROX, TAMRA, TET, TRIT (tetramethyl rhodamine isothiol), Tetramethylrhodamine, and Texas Red. These and other labels are available from commercial sources, such as Molecular Probes (Eugene, Oreg.).

### Solid Supports

Solid supports are solid-state substrates or supports with which molecules (such as trigger molecules, e.g., lysine) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

In some embodiments, a solid-state substrate may be used. Solid supports contemplated of use can include any solid material with which components can be associated, directly or indirectly. These material include but are not limited to acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multi-well glass slide can be employed.

In certain embodiments, an array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, in some embodiments, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances. In accordance with these examples, components can be associated or immobilized on a solid support at any density. Components can be immobilized to the solid support at a density exceeding 400 different components per cubic centimeter. Arrays of components can have any number of components depending on the circumstances.

### Pharmaceutical Compositions

In certain embodiments, compositions of identified test compounds may be generated for use in a subject having a bacterial infection in order to reduce or eliminate the infection in the subject. In accordance with these embodiments, the compositions can be administered in a subject in a biologically compatible form suitable for pharmaceutical administration in vivo. By “biologically compatible form suitable for administration in vivo” is meant a form of the active agent (e.g., pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically effective amount of an antibody or nucleic acid molecule reactive with at least a portion of lysine riboswitch may vary according to factors such as the disease state, age, sex, and weight of the individual, and the ability of antibody to elicit a desired response in the individual. Dosage regimens may be adjusted to provide the optimum therapeutic response. For example, several divided doses may be administered daily or the dose may be proportionally reduced as indicated by the exigencies of the therapeutic situation.

In one embodiment, the compound (e.g., pharmaceutical chemical, nucleic acid molecule, gene, protein, antibody, etc of the embodiments) may be administered in a convenient manner such as by injection such as subcutaneous, intravenous, by oral administration, inhalation, transdermal application, intravaginal application, topical application, intranasal or rectal administration. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be inhaled in order to make the compound bioavailable to the lung.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound that stimulates or inhibits a lysine riboswitch by other than parenteral administration, it may be necessary to coat the compound with, or co-administer the compound with, a material to prevent its inactivation. Enzyme inhibitors include pancreatic trypsin inhibitor, diisopropylfluorophosphate (DEP) and trasylol. Liposomes include water-in-oil-in-water emulsions as well as conventional liposomes. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by various antibacterial and antifungal agents (i.e., parabens, chlorobutanol, phenol, ascorbic acid, thimerosal, and the like). In many cases, it will be preferable to include isotonic agents, for example, sugars, polyalcohols such as manitol, sorbitol, sodium chloride in the composition. A compound such as aluminum monostearate and gelatin can be included to prolong absorption of the injectable compositions.

Sterile injectable solutions can be prepared by incorporating active compound (e.g., a chemical that modulates the lysine riboswitch) in the required amount in an appropriate solvent with one or a combination of ingredients enumerated above, as required, followed by filtered sterilization. Generally, dispersions are prepared by incorporating the active compound into a sterile vehicle that contains a dispersion medium and other required ingredients from those enumerated above. In the case of sterile powders for the preparation of sterile injectable solutions, the preferred methods of preparation are vacuum drying and freeze-drying which yields a powder of the active ingredient (i.e., a chemical agent, antibody etc.) plus any additional desired ingredient from a previously sterile-filtered solution thereof.

When the active agent is suitably protected, as described above, the composition may be orally administered (or otherwise indicated), for example, with an inert diluent or an assimilable edible carrier. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active agent and the particular therapeutic effect to be achieved, and (b) the limitations inherent an active agent for the therapeutic treatment of individuals.

### EXAMPLES

The following examples are included to illustrate various embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

Riboswitches act as genetic regulatory elements through the interplay of two distinct domains in the 5′-untranslated region (5′-UTR) of an mRNA: the aptamer domain that directly binds a specific cellular metabolite and a downstream expression platform containing a secondary structural switch that determines whether the gene will be expressed.

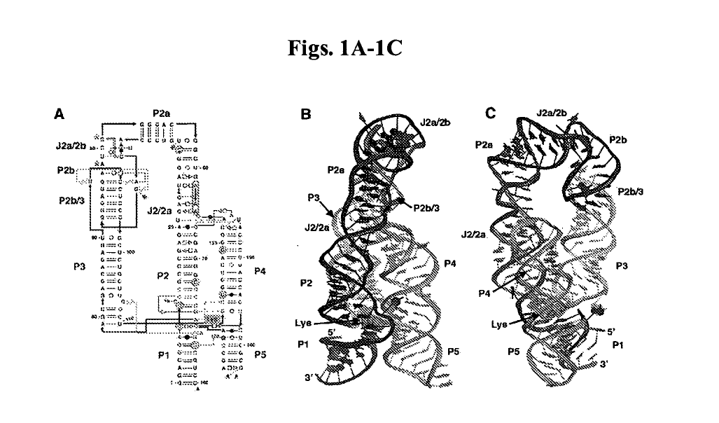

In one exemplary method, to understand the structural basis for lysine recognition and AEC resistance, the ligand binding domain of the lysine riboswitch was crystallized in the presence and absence of cognate ligand. A derivative of the sequence from the mRNA encoding the Thermotoga maritima β-aspartate semialdehyde dehydrogenase (asd), one of the first enzymes in the lysine biosynthetic pathway, readily yielded diffraction quality crystals in the presence of 1 mM lysine. This RNA contains all of the nucleotides whose identity is >90% conserved across the lysine riboswitch family (red, FIG. 1) (J. E. Barrick, R. R. Breaker, Genome Biol 8, R239 (Nov. 12, 2007), incorporated herein by reference). An iridium hexamine derivative yielded sufficiently high quality data from which an electron density map could be calculated (FIGS. 1A-1C and 2A-2D) and a model built comprising all 161 nucleotides and lysine. Data collection and refinement statistics for structures of both the liganded and unliganded RNA is presented in Table 1.

In one example, the 2.8 Å resolution structure of the RNA-lysine complex agrees well with previous genetic, biochemical, and phylogenetic analysis of the RNA. The global architecture of the RNA comprises three sets of coaxially stacked helices (P1-P2/2a, P2b-P2b/3-P3, and P4-P5) arranged roughly parallel to one another (FIGS. 1B and 1C). This mode of helical organization is a common theme in the structures of larger RNAs. A five-way junction that contains the bulk of the nucleotides with >90% conservation across phylogeny contains a binding site for a single lysine that is wedged between helix P1 and the J2/3 joining region.

Tertiary architecture of the RNA is dominated by formation of a three-helix bundle structure composed of the P2, P3, and P4 helices (FIG. 2A), stabilized via interactions mediated by their terminal loops. A kissing loop interaction is observed between L2 and L3 (FIG. 2B) that was identified as important for the ability of the B. subtilis lysC riboswitch to efficiently terminate transcription (see S. Blouin, D. A. Lafontaine, RNA 13, 1256 (2007)). Six contiguous Watson-Crick pairs are formed between the bases of the two loops to form P2b/3, which is coaxially stacked between P3 and the A39•A47 pair that forms P2b (in most other variants of this riboswitch, P2b comprises three base pairs (see J. E. Barrick, R. R. Breaker, Genome Biol 8, R239 (Nov. 12, 2007)).

Unlike other similar kissing loop interactions, it is further stabilized by a stacking interaction between G40 of L2 and U91 of L3 that is oriented perpendicular to the P2/3 helical axis (FIG. 2B). These two bases make a series of hydrogen bonding interactions between their Watson-Crick face and the major groove the central four base pairs of the P2b/3 helix. As this RNA is derived from a hyperthermophile, the additional dinucleotide “staple” may constitute an adaptation for function at elevated physiological temperatures. This type of adaptation has been observed in the in vitro selection of thermophilic ribozymes, where it was found that mutations that add new tertiary interactions or further stabilize existing ones are responsible for adaptation to function at high temperatures, rather than increasing the stability of the secondary structure F. (Guo, A. R. Gooding, T. R. Cech, RNA 12, 387 (March, 2006)).

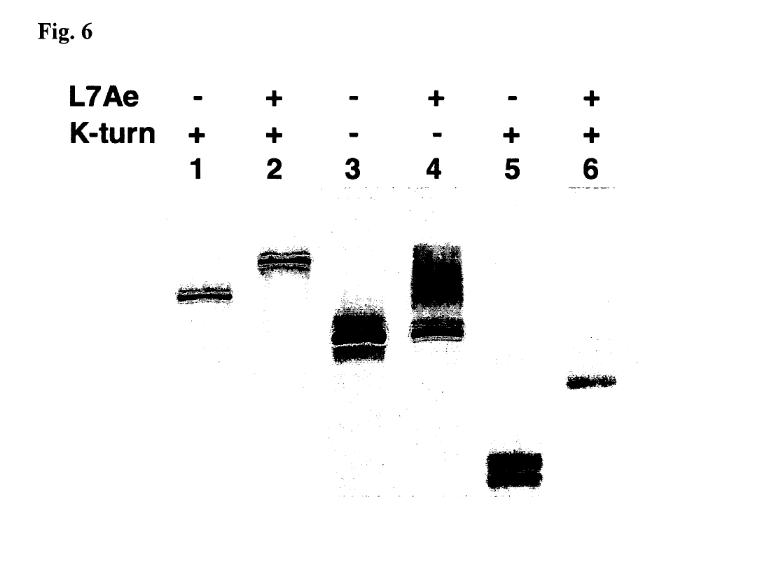

The ability of L2 to approach L3 from the opposite direction to form the kissing interaction is achieved by a ˜120° bend at J2a/2b using an internal loop motif that has not been previously observed. In the majority of other lysine riboswitches this turn has been demonstrated to be effected by a canonical kink-turn motif, and despite significant differences in their base interactions, they appear to effect a similar type of kink. To further verify that J2a/2b does not form a canonical kink-turn motif, we examined the ability of L7ae, a kink-turn binding protein, to specifically interact with the T. maritima asd lysine riboswitch using a native electrophoretic mobility shift assay. While the H. influenzae lysine and T. tencongensis SAM-I riboswitches that each contain a kink-turn motif specifically form a higher mobility complex with L7ae, the T. maritima lysine riboswitch does not (FIG. 6). Thus, while the majority of the aptamer domain is highly conserved, some elements of the peripheral region of the lysine riboswitch have evolved differing solutions to the stabilization of a common global architecture reflecting the modular nature of RNA structure (see N. B. Leontis, A. Lescoute, E. Westhof, Curr Opin Struct Biol 16, 279 (June, 2006)).

The second element stabilizing the three-helix bundle is an interaction between the terminal pentaloop of P4 and an internal loop motif adjacent to the sarcin/ricin motif between P2 and P2a. The pentaloop of P4 contains two conserved adenine residues (FIG. 1) that form part of a loop structure homologous to a standard GNRA tetraloop motif (FIG. 2C) that has been previously observed in the N-protein/boxB RNA complex (see P. Legault, J. Li, J. Mogridge, L. E. Kay, J. Greenblatt, Cell 93, 289 (Apr. 17, 1998)). Rather than docking with another helix using the sugar edge of the three stacked adenosine residues as observed for most tetraloop-mediated interactions (see P. Nissen, J. A. Ippolito, N. Ban, P. B. Moore, T. A. Steitz, Proc Natl Acad Sci USA 98, 4899 (Apr. 24, 2001)), the adenine bases interact with the minor groove of P2 using their Watson-Crick faces (FIG. 2C). Unusually, A123 forms the central base of a U21•A123•G65 base triple that anchors the interaction (FIG. 2D). Additionally, A124•G66•A20 triple and A126(N1)-G66(O2′) complete the pentaloop-receptor interaction. Bases in L4 and P2 that are involved in this interaction are the most conserved nucleotides outside of the five-way junction, indicating that this interaction is important to formation of the functional riboswitch.

FIGS. 1A-1C. represent exemplary structures of a lysine riboswitch. (A) Secondary structure of the T. maritima lysine riboswitch reflecting the tertiary structure of the RNA. Base pairing interactions are shown using the nomenclature of Leontis and Westhof (see Leontis and Westhof A. Wachter et al., Plant Cell 19, 3437 (November, 2007)). Circles denote interactions involving the Watson-Crick face, squares the Hoogsteen face, and triangles the sugar edge. Dashed lines denote interactions that do not fall into one of the standard pairing interactions. Nucleotides shown in red are >90% conserved across phylogeny and positions where mutations confer resistance to AEC are circled in blue (blue asterisks denote approximate positions). The structure is divided into three sets of coaxial stacks, defined as P1-P2/2a, P2b-P2b/3-P3, and P4-P5. (B) Cartoon diagram of the tertiary structure of the lysine riboswitch with each of the three stacks colored as designated in (A). Lysine is shown represented as van der Waals spheres. (C) 90° rotation of the perspective shown in (B).

FIGS. 2A-2D represent exemplary tertiary structural elements in the lysine riboswitch. (A) Top view of the riboswitch, as compared to perspective in FIG. 1B emphasizing the packing of the P2, P3, and P4 helices. (B) Molecular details of the kissing interaction between L2 and L3 to form P2b/3. The single base pair constituting a truncated P2b is at the top of the helix (A39•A47), followed by six consecutive Watson-Crick pairs and flanked by the closing U90•G98 pair in P3. A dinucleotide stack (G40, U91) makes hydrogen bonding contacts (grey dashes) to the central four base pairs in the major groove. (C) Cartoon of the L4 pentaloop docking with the minor groove of J2/2a. The non-canonical pairs in J2/2a contacted by the pentaloop are denoted as well as a phylogenetically conserved sarcin-ricin domain (SRD) motif that flanks the pentaloop docking site. (D) An unusual base triple in the pentaloop-J2/2a interaction in which the first adenosine residue (A123) of the loop partially invades into the J2/2a helix.

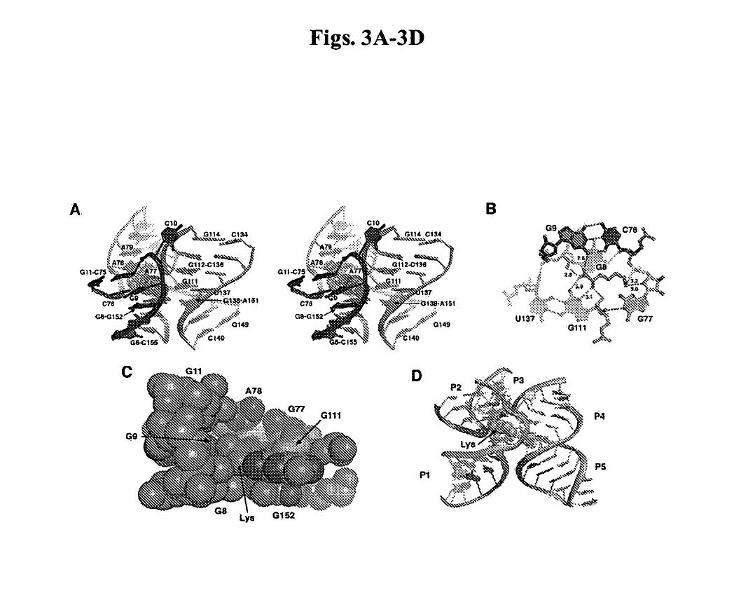

FIGS. 3A-3D represent exemplary lysine recognition by the five-way junction. (A) Stereo view of the binding pocket with lysine using the same color scheme as in FIG. 1. The van der Waals surface of lysine is represented with dots. Lysine is sandwiched between the minor grooves of the P1-P2 and P4-P5 stacks. (B) Details of the hydrogen bonding interactions between lysine and the RNA. The distances of the bonds between lysine and RNA are given in ångstrüms. (C) Van der Waal sphere representation of the lysine binding pocket emphasizing that lack of close packing of G77 and A78 on top of the methylene groups in the side chain. (D) Cartoon of the lysine binding pocket with ligand dependent cleavages as observed by in line probing highlighted in red. Regions of protection correspond to the joining region between P2 and P3 (J2/3), the 5′-side of P5, and the 3′-side of P1.

The ligand binding pocket is contained within the core of the five-way junction motif, sitting between P1 helix and J2/3 and flanked by the first base pairs of the P2 and P4 helices (FIG. 3A). The carboxylate group of lysine forms a set of hydrogen bonds with the N2 amino groups of the G137•U111 wobble pair and the G9-C76 Watson-Crick pair and the 2′-hydroxyl group of G8 (FIG. 3B). Further contacts to the N3 and O2′ atoms of G137 are made by the α-amino group of lysine. The ε-amino group of lysine is recognized by a combination of electrostatic and hydrogen bonding interactions within a pocket that places it close to the non-bridging phosphate oxygen of G77 (3.2 Å distance) along with the O4 oxygen atom of the ribose sugar (3.0 Å distance). Additional contacts are mediated between lysine and G111 and G152 by a well ordered solvent molecule (atomic displacement factor (ADP) of this water is 39.2 as compared to the ε-amino nitrogen's 35.1). The relatively small size of the ε-amino pocket near G77 precludes efficient recognition by homoarginine and N⁶-trimethyl-L-lysine. This may be the basis for discrimination between lysine and arginine in the cell.

Discrimination between lysine and other closely related compounds is effected through indirect recognition of the methylene linker of the side chain. The lysine side chain is bound in an extended conformation that allows it to span the two sites of interaction of the polar atoms, consistent with the ability of a lysine analog that contains a trans-double bond between the γ- and δ-carbons (see K. F. Blount, J. X. Wang, J. Lim, N. Sudarsan, R. R. Breaker, Nat Chem Biol 3, 44 (January, 2007)). Conversely, compounds containing shorter or longer side chains (L-ornithine and L-α-homolysine, respectively) are not efficiently bound because their side chain is of the incorrect length to allow the proper contacts between all of the polar atoms of lysine and the RNA. The hydrophobic methylene groups are primarily contacted through stacking interactions between A78 and the G8•G152 pair (FIG. 3C). However, the methylene groups are tightly packed against the RNA, particularly with the G77 and A78 (FIG. 3C), explaining the ability of lysine derivatives that contain modifications at the γ-position, as in L-3-[(2-aminoethyl)-sulfonyl]-alanine (AESA) and the antimetabolite S-(2-aminoethyl)-L-cysteine (AEC) to bind reasonably well to the RNA (7-fold and 30-fold lower affinity than lysine, respectively) (see N. Sudarsan, J. K. Wickiser, S. Nakamura, M. S. Ebert, R. R. Breaker, Genes Dev 17, 2688 (Nov. 1, 2003); and Blount, 2007). This behavior has also been observed in the TPP riboswitch, in which the central thiazole ring that is only moderately contacted can be significantly altered with little effect on ligand binding affinity or specificity (see for example, A. Serganov et al., Nature, (May 21, 2006); and S. Thore et al, Science 312, 1208 (May 26, 2006)).

Like other riboswitches, lysine is completely buried within the core of the five-way junction (100% solvent inaccessible), implying a ligand-dependent folding event concurrent with binding. Mapping of changes of magnesium-induced backbone strand scission of the RNA of the bound and unbound states using in-line probing (see for example Sudarsan et al., 2003) reveals two distinct sites of structural changes (red, FIG. 2D). The first is centered about J2/3, adjacent to the ε-amino pocket, suggesting that this might be the flexible “lid” that folds over the ligand, similar to what is observed in the guanine riboswitch's three-way junction (see C. D. Stoddard, R. T. Batey, ACS Chem Biol 1, 751 (Dec. 15, 2006)). A second site of protection is observed where the 5′-strand of P5 contacts the 3′-side of P1. This is striking in that ligand induced stabilization of the 3′-side of the P1-helix in a number of riboswitches is believed to be a crucial feature of their ability to determine the outcome of the downstream secondary structural switch in the expression platform; the lysine riboswitch appears to fit this trend.

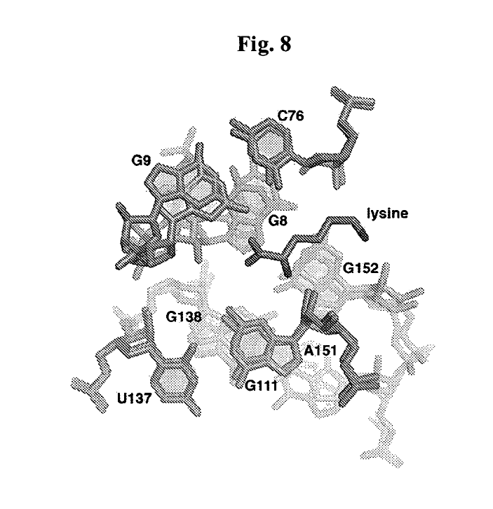

Thus, while solution probing indicates a different conformation in the RNA around the binding pocket in the absence of ligand, lattice contacts apparently provide sufficient energy to drive the RNA into the bound conformation. Therefore, in another exemplary method, to further examine the nature of the unbound form of this RNA and potential ligand induced conformational changes, it was crystallized in the absence of lysine. The RNA crystallized under the same conditions and in the same space group and the resulting structure is nearly identical to the complexed form (FIG. 7A), with only minor differences between the two structures in the positioning of the 5′-side of the P1 helix (FIG. 7B). This finding suggests that the global architecture is largely formed in the absence of ligand. Examination of the binding pocket reveals that positioning of some of the nucleotides are perturbed by 2-3 Å, but the overall pattern of base interactions remains the same (FIG. 8). This suggests that the energy difference between the free and bound conformations of the aptamer domain may be quite small, explaining why many riboswitches bind their targets with high affinity (nM) despite clearly coupling the ligand binding to allosteric changes in the RNA.

There are a number of mutations in the B. subtilis (see for example A. Wachter et al., Plant Cell 19, 3437 (November, 2007)) riboswitch that confer resistance to the antimetabolite AEC. A recent study revealed that the presumed loss of lysine-dependent regulation of expression of lysine biosynthetic genes results in a increased concentration of intracellular lysine that allows AEC to be effectively competed from its target, LysRS. Many of the mutations map with the five way junction, abrogating direct contacts with lysine. However, there are others observed in the distal regions of the P2 and P4 helix, distant from the lysine binding pocket (FIG. 1A). These mutations instead may either promote the formation of alternative structures that are binding-incompetent or decrease the rate at which the RNA is able to fold into a productive structure. In either case, since transcriptional regulation by riboswitches has a short temporal window in which to direct formation of the secondary structural switch, this result in a significant fraction of the RNA being incapable of rapidly binding lysine and thus promoting expression. This underscores the central importance of RNA folding processes in the biological function of riboswitches.

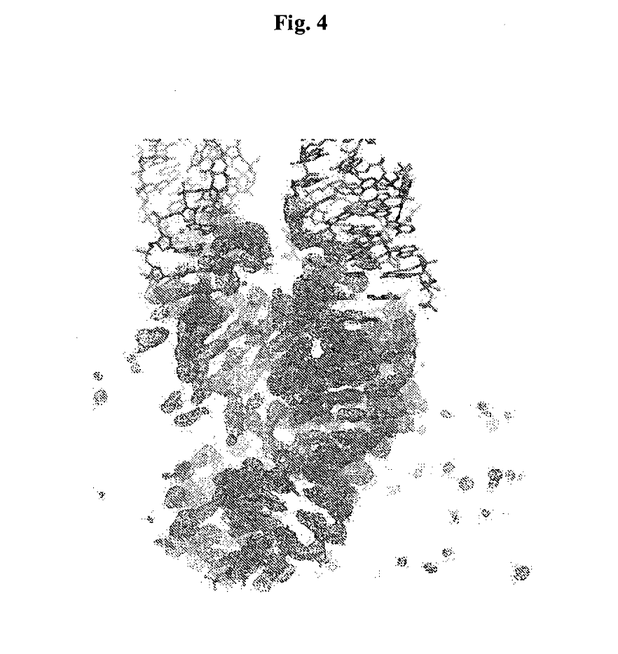

Table 1 illustrates exemplary crystallographic statistics. FIG. 4 represents an experimental electron density map. A portion of the experimental electron density map (blue mesh) unbiased by model phases contoured at 1.5σ. The final model (sticks) is overlaid on the map to provide perspective.

FIGS. 5A and 5B represent exemplary maps of the ligand binding pocket. (A) Final 2Fo-Fc map contoured at 1.0σ around the nucleotide residues that define the binding pocket and lysine. (B) Simulated annealing omit map in which residues 76, 77, 111 were omitted along with lysine. Note that the density around the ligand remains defined for the entire amino acid and its positioning within the pocket is unambiguous.

FIG. 6 represents an exemplary mobility shift assay of riboswitches with protein L7Ae. The lysine riboswitch does not require a kink turn (k-turn) for function. L7Ae specifically recognizes the RNA k-turn motif as seen for the lysine riboswitch from H. influenzae (lanes 1 and 2) and the SAM-I riboswitch from T. tencongensis (lanes 5 and 6). The lysine riboswitch from T. maritima reported here (lanes 3 and 4) shows that the k-turn motif is absent. In the two RNAs containing a known kink turn motif, a clear shift in mobility is observed with the addition of L7Ae (lanes 2 and 4).

FIGS. 7A and 7B represent exemplary superposition of free and bound lysine riboswitch. (A) Superpositioning of the free (orange) and bound (green) structures of the lysine riboswitch using the Theseus alignment program (see D. L. Theobald, D. S. Wuttke, Bioinformatics 22, 2171 (Sep. 1, 2006)). The two structures superposition with a maximum likelihood r.m.s.d. of 0.08 Å (classical pairwise r.m.s.d. is 0.70 Å). (B) Map of the estimated variance between the two structures in atomic coordinates between the two structures; blue represents low variance (<1 Å²) and red denotes high variance (>10 Å²).

FIG. 8 represents exemplary details of superposition of the binding pocket. Close up of the lysine binding pocket with the superposition of the free (orange) and bound (green; lysine in magenta) RNA. The largest differences around the binding pocket are in G9, and the G8•G152, G139•A151 pairs that form the floor of the pocket.

### Methods and Materials

### RNA Preparation.

A 161 nucleotide construct consisting of the sequence for the riboswitch aptamer domain from the asd gene of T. maritima was constructed by PCR using overlapping DNA oligonucleotides (Integrated DNA Technologies). The resulting dsDNA fragment contained sites for restriction digest with enzymes EcoRI and NcoI, and following digestion this piece was ligated into plasmid vector pRAV 12, which is designed for denaturing purification of RNA (see J. S. Kieft, R. T. Batey, RNA 10, 988 (June, 2004) incorporated herein in its entirety). The cloned sequence was subsequently verified before use in transcription. Transcription template (dsDNA) for large scale reactions was prepared by PCR using primers directed against the T7 promoter (SEQ ID NO:1 5′, GCGCGCGAATTCTAATACGACTCACTATAG, 3′) and the HdV ribozyme contained in the pRAV12 plasmid (SEQ ID NO:2 5′, GAGGTCCCATTCATTCGCCATGCCGAAGCATGTTG, 3′). This ribozyme catalyzes site specific cleavage of the RNA transcript that homogenizes the 3′ end of the riboswitch construct leaving a single base overhang (Kieft et al., 2004). RNA was transcribed in 12.5 mL reactions containing 30 mM Tris-HCl (pH 8.0), 10 mM DTT, 0.1% Triton X-100, 2 mM spermidine-HCl, 4 mM each NTP (Sigma and Research Products Inc.), 24 mM MgCl₂, 0.25 mg/mL T7 RNA polymerase, 1 mL of ˜0.5 μM template, and 0.32 unit/mL inorganic pyrophosphatase (Sigma) to inhibit formation of insoluble magnesium pyrophosphate. The transcription reaction was allowed to proceed for two and one half hours at 37° C., after which the reactions were placed at 70° C. for 15 minutes to enhance cleavage rate of the HdV ribozyme. RNA was then ethanol precipitated at −20° C. overnight and subsequently purified by denaturing PAGE (12% polyacrylamide, 1×TBE, 8 M urea). The band pertaining to the proper size was visualized by UV shadowing, excised, and electroeluted overnight in 1×TBE to extract the RNA from the gel. The eluted fraction was exchanged three times into 10 mM Na-HEPES at pH 7.0, 2 mM lysine buffer using a 10,000 MWCO centrifugal filter and then refolded by heating to 95° C. for three minutes followed by snap cooling on ice. The refolded RNA was then exchanged three times into 10 mM Na-HEPES pH 7.0, 5 mM MgCl₂, and 2 mM lysine before storage. For the free state, the refold was done in the lysine supplemented buffer to promote proper folding and exchanged three times into 10 mM Na-HEPES pH 7.0, 5 mM MgCl₂ followed by overnight dialysis into 1 L of this buffer. Typical yields of 250 mL of 400 mM were obtained as judged by absorbance at 260 nm and the calculated extinction coefficient. RNA was stored at 4° C. until use.

### Crystallization.

The riboswitch was crystallized by the hanging drop vapor diffusion method at concentrations of 1 mM lysine, or in the absence of lysine for the free state crystals. Drops were set up by mixing 1 μL of RNA with 1 μl, of a mother liquor solution consisting of 60 mM iridium hexaammine, 2 M Li₂SO₄, 5 mM MgCl₂, and 10 mM Na-HEPES pH 7 to obtain the heavy atom derivative crystals. The iridium hexaammine used in these experiments was prepared as described previously (R. K. Montange, R. T. Batey, Nature 441, 1172 (Jun. 29, 2006) incorporated herein in its entirety). The same conditions were used to grow the free state crystals. Crystals were obtained within 24 hrs and required no additional cryoprotection agent due to the high ionic strength of the crystallization buffer. Crystals were looped with 0.2-0.3 μm loops then flash-frozen in liquid nitrogen before data collection.

### Data Collection.

Single wavelength anomalous diffraction (SAD) data for the bound state iridium hexaammine derivative crystal was collected on beamline 8.2.1 at the National Synchrotron Light Source in New York using X-rays with λ=1.1050 Å at the Ir absorption peak, integrated, and scaled using D*TREK (see J. W. Pflugrath, Acta Crystallogr D Biol Crystallogr 55, 1718 (October, 1999)). The crystals belong to the P3₂ space group (a=119.823 Å, b=119.823 Å, c=58.744 Å, a=b=90°, c=120°) and have one molecule per asymmetric unit. All data used in phasing and refining came from a single derivative crystal. Data for the unliganded structure were collected at the Cu-Kα wavelength (1.5418 Å).

### Phasing and Structure Determination.

Phases were determined by single wavelength anomalous diffraction (SAD) using data that extended to 2.8 Å. The peak and inflection wavelength datasets were merged and scaled using the SHELX software package (see A. T. Brunger et al., Acta Crystallogr D Biol Crystallogr 54, 905 (Sep. 1, 1998) and G. M. Sheldrick, Acta Crystallogr A 64, 112 (January, 2008)) and Patterson maps were then calculated for both space groups P3₁ and P3₂. From the maps it was determined that there were four possible iridium sites within the unit cell with reasonably high occupancy. A CNS heavy-atom search for four possible sites was then carried out in both space groups, and both space groups yielded 94 possible solutions. The best of these were used to calculate predicted Patterson maps, which showed peaks that correlated very well with those seen in the original maps in all four Harker sections. The best solution sites were used to calculate phases in SHELXD. The resulting density map for P3₁ was uninterpretable, whereas the map for P3₂ displayed clear density for the helical structures that are characteristic of RNA. The phasing solution found by SHELXE had a figure of merit of 0.6332 which was further improved to 0.8846 following a round of density modification with the solvent level set to 0.46. The phasing power at the peak wavelength was 3.3 with a R_{cultis} of 0.39 (acentric).

The model was built in Coot (see Coot P. Emsley, K. Cowtan, Acta Crystallogr D Biol Crystallogr 60, 2126 (December, 2004). and refined in PHENIX (P. D. Adams et al., Acta Crystallogr D Biol Crystallogr 58, 1948 (November, 2002) in iterative rounds. The RNA nucleotides were placed in the first round, the iridium hexaammines were placed in the second round. This model was taken through multiple rounds of simulated annealing before the addition of lysine to the binding pocket. Structure, parameter, and topology files for iridium hexaammine were generated using the ELBOW feature in the PHENIX software suite; the parameters for lysine were already loaded in PHENIX. The density for lysine was unambiguous after simulated annealing making placement of this molecule straight forward. This was followed by one round of water-picking carried out by the PHENIX ordered solvent protocol. Waters were chosen based on peak size in an anomalous difference map. The minimum was set to 2.5 s with the additional parameters that the B-factor could be no greater than 120, and the peak must be within hydrogen bonding distance of the oxygens and nitrogens in the RNA. Each round of model-building was followed by a simulated annealing run and B-factor refinement using PHENIX. R_{free} was monitored in each round to ensure that it was dropping. Sugar puckers were restrained in most cases to C3′ endo, except for residues which were restrained to C2′ endo. Figures were prepared using Ribbons 3.0 (see M. Carson, Methods Enzymol 277, 493 (1997)). and Pymol (see W. L. Delano. (DeLano Scientific, San Carlo, Calif., USA, 2002)).

### Chemical Probing Using Selective 2′-Hydroxyl Acylation and Primer Extension (Shape) Chemistry.

SHAPE chemistry provides a means to assess the conformational dynamics or degree of 2′-endo constrained puckering of every nucleotide in the RNA backbone (see Merino et al., J Am Chem Soc 127, 4223 (Mar. 30, 2005)). The DNA sequences of the riboswitch aptamer domains from the lysC gene in B. subtilis and the T. maritima construct used in the crystallographic studies were chosen for this analysis. The B. subtilis sequence was truncated in the P5 region to match the length of the T. maritima sequence for the sake of consistency. The 5′ and 3′ structure cassettes were appended to these sequences as described previously (see Wilkinson et al., Nat Protoc 1, 1610 (2006)). Modifications were carried out at 667 μM Lysine and 10 mM MgCl₂

TABLE 1
Data collection, phasing, and refinement statistics (SIRAS)

| | RNA-ligand complex | |
|---|---|---|
| | Ir-hexamine | Free RNA |
| Data collection | | |
| Space group | P3₂ | P3₂ |
| Cell dimensions | 119.82, 119.82, 58.74 | 120.19 120.19 58.25 |
| a, b, c (°) | 90, 90, 120 | 90, 90, 120 |
| | Peak | |
| Wavelength | 1.1050 Å | 1.5418 Å |

| | | | | |
|---|---|---|---|---|
| Resolution (Å) | 40.0-2.8 | (2.91-2.8)* | 19.70-2.95 | (3.06-2.95) |
| R_{sym} or R_{merge} | 8.4% | (34.8%) | 9.0% | (35.5%) |
| l/s/ | 17.9 | (4.4) | 10.2 | (3.4) |
| Completeness (%) | 99.5 | (96.6) | 99.5 | (100) |
| Redundancy | 5.2 | (3.7) | 3.62 | (3.63) |
| Refinement | | | | |
| Resolution (Å) | 32.6-2.8 | (2.87-2.8) | 17.11-2.95 | (3.02-2.95) |
| No. reflections | 23986 | (97.7%) | 19610 | (99.4%) |

| | | |
|---|---|---|
| R_{work/}R_{free} | 18.20/20.86 | 18.61/22.04 |
| No. atoms | 3631 | 3547 |
| RNA | 3491 | 3491 |
| Ligand | 10 | N/A |
| Water | 55 | 56 |
| B-factors | 54.61 | 54.92 |
| RNA | 47.22 | 55.12 |
| Ligand/ion | 35.76 | N/A |
| Water | 39.14 | 42.87 |
| r.m.s deviations | | |
| Bond lengths (Å) | 0.005 | 0.004 |
| Bond angles (°) | 1.251 | 1.239 |
| Maximum likelihood | 0.32 | 0.43 |
| coordinate error (Å) | | |

Data was collected from a single crystal.
*Highest resolution shell is shown in parenthesis.

Some of the work described in this application was published in Garst, et al. “Crystal Structure of the Lysine Riboswitch Regulatory mRNA Element”, J. Biol. Chem. 283(33): 22347-223.51 (published Jun. 12, 2008), the contents of which article (including the supplemental tables and figures available on the on-line version at http://www.jbc.org) are incorporated herein by reference. The atomic coordinates and structure factors for the crystal structure of the lysine riboswitch (code 3D0U, depicting riboswitch bound to lysine, and code 3D0X, depicting unbound riboswitch) have been deposited in the Protein Data Bank, Research Collaboratory for Structural Bioinformatics, Rutgers University, New Brunswick, N.J. (http://www.rcsb.org/), and are incorporated herein by reference.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for identifying a compound that associates with a lysine riboswitch comprising the steps of:
modeling at least one portion of the lysine riboswitch atomic structure depicted in at least FIG. 3 with a test compound; and
determining an association between the test compound and the lysine riboswitch atomic structure.

2. The method of claim 1, further comprising determining that the test compound reduces bacterial gene expression.

3. The method of claim 1, further comprising determining that the test compound induces bacterial gene expression.

4. The method of claim 1, wherein the association determination step comprises determining at least one of a minimum interaction energy, a binding constant, a dissociation constant, or a combination thereof, for the test compound with the modeling of at least one portion of the lysine riboswitch atomic structure.

5. The method of claim 1, wherein the association determination step comprises determining the interaction of the test compound with one or more nucleotides of the lysine riboswitch comprising G9, C76, G77, G111, U137 or combinations thereof.

6. The method of claim 1, wherein the association determination step further comprises determining an interaction of the test compound with a lysine moiety comprising a carboxylate and/or amino moieties or combination thereof.

7. The method of claim 1, wherein the association determination step further comprises determining an interaction of the test compound with a nucleotide of the lysine riboswitch atomic structure comprising G9, C76, G77, G111, U137 or a combination thereof.

8. The method of claim 1, wherein the association determination step further comprises determining an interaction of the test compound with a P1 helix and J2/3 of the lysine riboswitch atomic structure.

9. A method of regulating gene expression in a cell by modulating an mRNA, the method comprising the steps of administering a lysine riboswitch modulating compound to the cell to modulate the lysine riboswitch activity of the mRNA.

10. The method of claim 9, wherein gene expression is stimulated.

11. The method of claim 9, wherein gene expression is inhibited.

12. The method of claim 9, wherein the lysine riboswitch modulating compound forms a complex with the lysine riboswitch decreasing the formation of an antiterminator element by the mRNA.

13. The method of claim 10, wherein the cell is a bacterial cell.

14. The method of claim 14, wherein the bacterial cell is a Gram-negative bacterial cell.

15. A lysine riboswitch, wherein one or more of nucleotides G9, C76, G77, G111, or U137 are modified.

16. The method of claim 15, wherein interaction with a lysine riboswitch having the one or more modified nucleotide causes an increase in gene expression in a cell.

17. The method of claim 15, wherein interaction with a lysine riboswitch having the one or more modified nucleotide causes a decrease in gene expression in a cell.

18. The method of claim 15, wherein interaction with a lysine riboswitch having the one or more modified nucleotide causes a decrease in sulfur production in a cell.

19. A composition comprising a compound that associates with at least a portion of the lysine riboswitch atomic structure depicted in FIG. 3B, wherein the association includes compound interaction with at least one of nucleotides G9, C76, G77, G111, U137 and wherein the composition is capable of modifying lysine riboswitch activity in a bacterial organism.

20. The composition of claim 19, wherein the composition further comprises a pharmaceutically acceptable excipient.

21. A composition comprising at least 80% of a conserved nucleotide sequence of a lysine riboswitch core depicted in FIG. 1A and 80% or more of nucleotides depicted outside of a conserved region depicted in FIG. 3B.

22. The composition of claim 21, further comprising a nucleotide sequence depicted in FIG. 5A.

23. Computer software for modeling the interaction between a lysine riboswitch and a ligand.

24. A computer comprising the software of claim 23.

25. A method of screening compounds comprising using a computer to model the atomic structure of the lysine riboswitch, the atomic structure of a test compound and the interaction between them.
